(19) United States
(12) Patent Application Publication
(10) Pub. No.: US 2007/0055124 A1
(43) Pub. Date: Mar. 8, 2007

(54) Method and system for optimizing left-heart lead placement

(76) Inventors: Raju R. Viswanathan, St. Louis, MO (US); William Flickinger, Twin Cities, MN (US)

Correspondence Address: HARNESS, DICKEY, & PIERCE, P.L.C, 7700 BONHOMME, STE 400, ST. LOUIS, MO, 63105, US

(21) Appl. No.: 11/218,287

(22) Filed: Sep. 1, 2005

(51) Int. Cl. A61B 5/05

(52) U.S. Cl. 600/407

(57) ABSTRACT

A system for identifying locations for pacing the heart, the system comprising a pacing electrode, a remote navigation system for positioning the pacing electrode at each of a plurality of locations; a system for determining Pressure-Volume loop data resulting from pacing at each location; an ECG system, a phrenic nerve stimulation detection system, and a means of identifying at least one preferred location based upon at least the Pressure-Volume loop, ECG, and phrenic nerve stimulation data at each location. A method of identifying locations for pacing the heart, the method comprising: navigating a pacing electrode to each of a plurality of locations in the heart; pacing the heart at each of the plurality of locations; and assessing the effectiveness of the pacing at each location by measuring cardiac blood flow and cardiac wall strain.

### CROSS REFERENCE TO RELATED APPLICATION

This application is a Continuation In Part of and claims priority from U.S. patent application Ser. No. ______, filed Aug. 24, 2005, entitled Methods and Apparatus for Steering Medical Devices in Body Lumens [Attorney Docket Number 5236-000602], which claims priority from U.S. Provisional Patent Application Ser. No. 60/604,101, filed Aug. 24, 2004, Methods and Apparatus for Steering Medical Devices in Body Lumens, and U.S. patent application Ser. No. 10/448,273, filed May 29, 2003, entitled Remote Control of Medical Devices Using a Virtual Device Interface; which claims priority of U.S. Provisional Patent Application No. 60/401,670, filed Aug. 6, 2002, entitled, Method and Apparatus for Improved Magnetic Surgery Employing Virtual Device Interface, and of U.S. Provisional Patent Application Ser. No. 60/417,386, filed Oct. 9, 2002, Method and Apparatus for Improved Magnetic Surgery Employing Virtual Device Interface, the disclosures of all which are incorporated herein by reference.

### BACKGROUND OF THE INVENTION

This invention relates to the placement of pacing leads in the heart, and in particular to method of optimizing the placement of leads in the heart.

Left heart lead placement is fraught with difficulties including accessing the Coronary Sinus, sub-selecting veins, and finding an implant site in the coronary venous structure that provides an ideal pacing response without phrenic nerve stimulation. The structure of the coronary venous system, coupled with the conventional tools available, often make lead placement a time-consuming part of the overall implant procedure. The lead implant procedure is further complicated by the fact that QRS width has been shown to be an imprecise predictor of outcome.

### SUMMARY OF THE INVENTION

Generally, embodiments of the methods of the present invention provide for improved placement of pacing leads. Various embodiments of the methods of the present invention optimize lead placement by providing various measures for predicting lead implantation success, and for sensing bad locations including those that must be ruled out due to unacceptable side-effects of pacing (such as phrenic nerve stimulation). Some embodiments of the methods of this invention provide an automated method for device navigation and lead implant site selection.

### BRIEF DESCRIPTION OF THE DRAWINGS

FIG. 1 is a schematic diagram of one possible embodiment of a system for optimizing lead placement in accordance with the principles of this invention;

FIG. 2 is a schematic diagram of a display from one possible embodiment of a system for optimizing lead placement in accordance with the principles of this invention;

FIG. 3 is a block diagram of an algorithm of the heuristic decision tree employed in one possible embodiment of the method of optimizing lead placement in accordance with the principles of this invention.

### DETAILED DESCRIPTION OF THE PREFERRED EMBODIMENTS

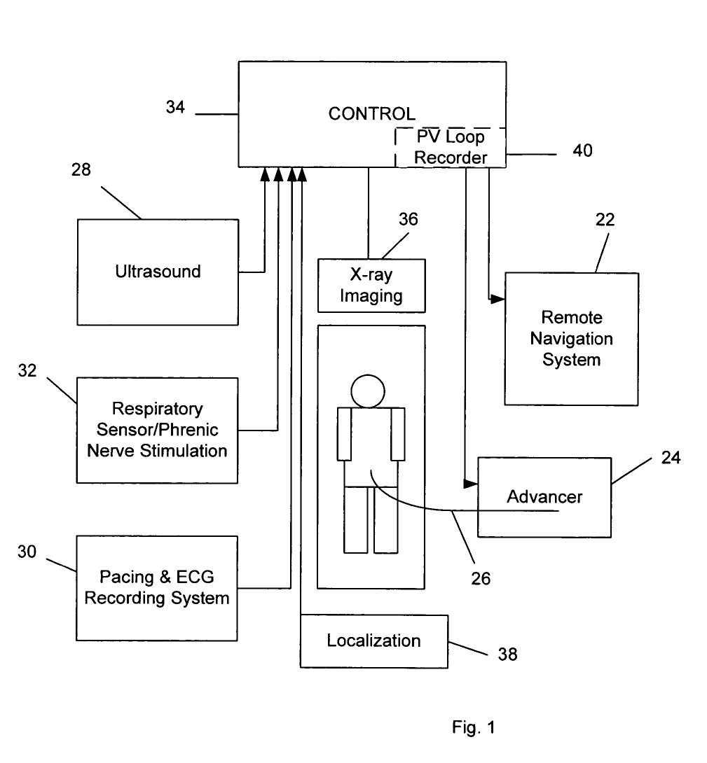

This invention relates to the navigation and placement of pacing leads in the heart. Embodiments of this invention provide a system for, and methods of, optimally placing such pacing leads. A preferred embodiment of a system for placing pacing leads in accordance with this invention is indicated generally as 20 in FIG. 1. The system 20 comprises a remote navigation system 22. This remote navigation system 22 preferably has the ability to remotely orient the distal end of a medical device 24 such as a guide wire or catheter, and advance the end to a selected location. One example of such a remote navigation system is the Niobe® remote magnetic navigation system available from Stereotaxis, Inc., St. Louis, Mo., which uses external source magnets to create a magnetic field in a selected direction in the operating region in a subject. This magnetic field acts on a magnetically responsive element at the distal end of the medical device to orient the medical device in a selected direction. An advancer acting on the proximal end, advanced the distal tip in the selected direction. Another example of a remote navigation system is a mechanical system which uses a mechanically operated guide sheath to orient the distal end portion of a guide wire or catheter. An advancer 26 acting on the proximal end of the guide wire or catheter 24 advances the guide wire or catheter through the mechanically operated guide in the selected direction. While the description and drawings relate primarily to magnetic navigation systems, the invention is not so limited, and the systems and methods can be implemented with any remote navigation system.

The system 20 preferably also comprises an ultrasound echocardiography system 28 capable of imaging and measuring and recording blood flow properties of the heart. The ultrasound echocardiography system is preferably an external system, but could also be an esophageal system. The ultrasound echocardiography system 28 preferably allows the measurement of at least one of the flow rate, strain rate, and ejection volume of the heart in addition to displaying a real-time image of the heart. The ultrasound echocardiography system 28 can provide volume data to a pressure volume loop sensing system 40 which provides real-time pressure loop recordings with pressure data obtained, for example, from a catheter having a pressure transducer placed in a cardiac chamber. The system 20 may also include ECG system 30, which can be used for among other things to determine QRS width.

The system 20 may also include a sensor 32 for phrenic nerve stimulation during pacing. In one embodiment, the phrenic nerve stimulation sensor 32 consists of a pressure-sensitive pad commonly known as a Grasby pad that is connected to a pressure sensor with an output that can be displayed on a recording system. In another embodiment, the sensor 32 can be a piezo-electric sensor attached to a belt. In yet another, the sensor 32 can be a piezoelectric pad that is placed under the subject and can be used to detect respiration and/or cardiac output, while in still another embodiment the sensor 32 is a thermal sensor at the nose that is designed to sense the temperature changes due to inhalation and exhalation. In still another embodiment, the sensor 32 is a set of electrodes and amplifier designed to detect phrenic nerve or diaphragmatic activity. In still another embodiment, the sensor 32 is a nasal canula attached to a pressure sensor that detects the positive and negative pressures generated by respiration in the range of 0-15 cmH₂O. In yet another embodiment, it is an infrared sensor aimed at the airway. This list is not exhaustive, and in implementation the sensor 32 can be any current or subsequently developed sensor whose output correlates to phrenic nerve stimulation.

The system 20 may include any other devices for monitoring the heart or heart function or other physiologic response during pacing in order to determine the suitability of a particular lead placement. For example, in an alternate embodiment a magnetic resonance imaging system could be used to obtain the flow rate, cardiac volume and cardiac strain rate information. The system 20 may further comprise a control 34 that operates the remote navigation system 22 and the advancer 24 in response to inputs from various sensors including for example ultrasound system 28, ECG system 30, and phrenic nerve stimulation sensor 32.

The systems and methods of the preferred embodiments of the invention can employ a guide wire capable of pacing, a pacing lead that can be advanced over a guide wire, or a catheter with a packing electrode or carrying a pacing lead, to pace various sites in the heart to find the optimal pacing location according to predetermined criteria, within the coronary vasculature. In one embodiment of the method of this invention, the device (whether guide wire, catheter, or pacing lead) could be advanced and retracted manually. However, the device is preferably advanced remotely using a motorized system to advance and retract the device, either under the physician's control or completely automatically under the control of a processor.

In a preferred embodiment, the system 20 includes at least two fluoroscopic views from bi-plane fluoroscopy system 36 with a sufficient angular separation that would allow for the system to use edge-detection image analysis techniques to create a 3-D image of the coronary vasculature. Alternatively, a 3-D preoperative image, such as those obtained by CT or MRI, can be imported into the system for the same purpose. Given such three dimensional path information, the control 34 of remote navigation system 22 can use this three dimensional information to automatically navigate or steer a device, such as a guide wire, through the anatomy from a given starting location to any of a multiplicity of destination locations within the vasculature. Such a method of automated vascular steering with a remote surgical navigation system is taught in U.S. patent application Ser. No. ______, filed Aug. 24, 2005, entitled Methods and Apparatus for Steering Medical Devices in Body Lumens which claims priority from U.S. Provisional Patent Application Ser. No. 60/604,101, filed Aug. 24, 2004, Methods and Apparatus for Steering Medical Devices in Body Lumens, incorporated herein by reference. In one preferred embodiment, the destination locations are defined by a user by means of suitable markings defined on a pair of fluoroscopic images to define three dimensional points. In another preferred embodiment, the destination locations are selected from a subset of previously visited locations by the remote navigation system.

The guide wire or other medical device can be advanced and retracted by the physician based on the data that is displayed on a graphical user interface of the remote navigation system; alternatively the advancement and retraction of the device can be automated using advancer 24. For automated advancement and retraction devices 24 the system 20 may also include a localization system 38 for determining the location of the distal end of the medical device within the operating region in the subject. The localization system 38 can be an image processor which processes images of the operating region (either the ultrasound images or the fluoroscopic images) to localize the distal end of the medical device. The localization system 38 can also be an electromagnetic localization system, such as the Carto™ system available from Biosense Webster Inc. The invention is not limited to any particular localization system, and only requires sufficient information of the location of the distal tip of the device in order to permit safe, automated navigation.

In a preferred embodiment, a control 40 integrates data from a plurality of available sources, including volumetric flow rate, cardiac output volumes and cardiac strain rates from the ultrasound echocardiography imaging system 24, pressure data from a catheter incorporating a pressure transducer and placed within the patient anatomy or from a less invasive pressure measurement such as a piezoelectric mat or a trans-thoracic impedance-based pressure measuring device. The integration of ultrasonic image data with a remote navigation system is described in U.S. patent application Ser. No. 10/448,273, filed May 29, 2003, entitled Remote Control of Medical Devices Using a Virtual Device Interface; which claims priority of U.S. Provisional Patent Application No. 60/401,670, filed Aug. 6, 2002, entitled, Method and Apparatus for Improved Magnetic Surgery Employing Virtual Device Interface, and of U.S. Provisional Patent Application Ser. No. 60/417,386, filed Oct. 9, 2002, Method and Apparatus for Improved Magnetic Surgery Employing Virtual Device Interface (incorporated herein by reference) and in particular the integrated ultrasonic image data provides information about cardiac wall motion and blood flow rates. The integrated pressure and volume data is used in particular to determine the PV (Pressure-Volume) loop corresponding to the cardiac cycle. The area enclosed by this loop (suitably gated to the ECG signal) from ECG monitor 30 is a measure of cardiac work output, and thus cardiac efficiency. Additionally, the width of the ECG pulse can be recorded by connecting to an ECG recording system 30 such as the Prucka™ system manufactured by GE.

Different pacing locations can thus be compared in order to determine the ideal location or locations for lead implantation. This can be done in an automated manner by the remote navigation system. The electrode or lead can continuously pace the heart as it is advanced, retracted, redirected and advanced again down the various coronary vessels automatically by the system until a suitable location is found for lead placement. This automated remote navigation method is described in detail in U.S. patent application Ser. No. ______, filed Aug. 24, 2005, entitled Methods and Apparatus for Steering Medical Devices in Body Lumens which claims priority from U.S. Provisional Patent Application Ser. No. 60/604,101, filed Aug. 24, 2004, Methods and Apparatus for Steering Medical Devices in Body Lumens, (incorporated herein by reference). Ultrasound imaging data can be used to further optimize control of the device using a computational model, as described in U.S. patent application Ser. No. 10/448,273, filed May 29, 2003, entitled Remote Control of Medical Devices Using a Virtual Device Interface; which claims priority of U.S. Provisional Patent Application No. 60/401,670, filed Aug. 6, 2002, entitled, Method and Apparatus for Improved Magnetic Surgery Employing Virtual Device Interface, and of U.S. Provisional Patent Application Ser. No. 60/417,386, filed Oct. 9, 2002, Method and Apparatus for Improved Magnetic Surgery Employing Virtual Device Interface (incorporated herein by reference).

The Pressure-Volume loop can be used to evaluate pacing locations. The area Aᵢ inside the Pressure-Volume loop for each lead location (indexed by i) that is “interrogated” is automatically determined. In conjunction with this, the phrenic nerve activity data (in the form of respiration monitoring, thermal changes due to inhalation/exhalation, or electrode recordings) can also be recorded. Locations {j} at which the phrenic nerve activity is larger than a threshold value of a suitable variable (depending on measurement method) such as a respiration rate, thermal changes in inhalation/exhalation, or electrical activity recorded by electrodes, are removed from consideration. Of the remaining locations, the location k with the largest PV-loop area Aₖ is recommended by the remote navigation system as the ideal site for lead implantation. However this location may be geographically unfavorable (it may be difficult to navigate to, or difficult to secure a pacing lead, etc.) and may be rejected by the physician, in favor of an alternate point at which the PV loop area is smaller but at which the location is more geographically desirable.

In an alternate embodiment, the width of the ECG pulse at each location is also used to determine the optimal location. In this case, for the location with the largest-area PV-loop, an additional check is performed to ensure that the ECG pulse width lies within a certain pre-determined range of values. If it does not, the location with the next-largest PV-loop area is chosen, and so on. In one embodiment, the remote navigation system automatically navigates the lead to the thus-determined ideal location.

In other embodiments, the rate of change of pressure with respect to time can also be used as a criterion for selection of the optimal lead placement location.

In another embodiment, a set of heuristics that could be used by a user to pick an optimal location is displayed on the User Interface of the remote navigation system. The total heuristic data set may include some or all of the following data, including remote navigation system control variables together with other clinical data compiled on one screen including (any data item may be present or absent):

Pacing Threshold

Sensing amplitude

dP/dt of pressure waveform for EF measurements

PV loop for EF measurements

Echocardiogram for EF measurements

ECG QRS for width evaluation (goal <120 ms)

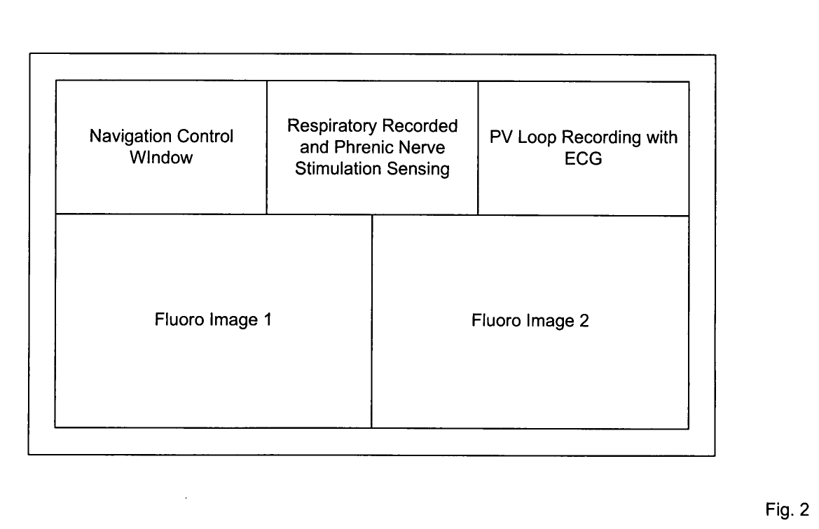

One possible layout of the display of a user interface for implementing methods and systems in accordance with the principles of this invention is shown in FIG. 2. In the particular embodiment shown in FIG. 2, some of the data detailed above is provided within the display. In a preferred embodiment, the remote navigation system is a magnetic navigation system. In this case the control variables are an externally applied magnetic field vector orientation and length of extension of the device. In another preferred embodiment, the remote navigation system is a mechanically actuated navigation system where the control variables could be pull-wire cable tensions, servo motor configurations, or the like.

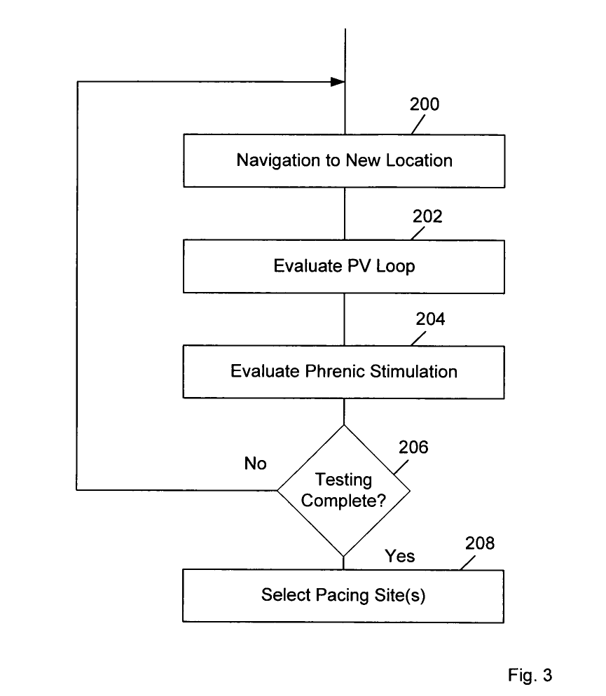

FIG. 3 shows a process flow diagram for the method described in this invention. At step 200 the pacing lead is navigated to a location, at step 202 pacing is initiated at the location, and the PV loop is evaluated. The PV loop can be determined using inputs from ultrasound system 28 and ECG system 30. At 204 the phrenic stimulation is evaluated. At 206, if the pacing is not complete, step 200 is repeated. If pacing is complete, then at 208 one or more pacing sites are identified based upon at least the PV loop and phrenic stimulation.

In an alternate preferred embodiment, a Magnetic Resonance Imaging system is used in place of the ultrasound echocardiography system in order to quantify cardiac volume output and flow rates, while the remote navigation system is actuated by mechanical or electrostrictive means.

While the above description details the use of magnetic and mechanical remote navigation systems, any other mode of remote actuation such as electrostrictive, hydraulic, or magnetostrictive or others known to those skilled in the art can be used as an actuation modality by the remote navigation system. Likewise, while some means of pressure measurement are described above, other methods of such measurement can also be used according to the teachings of the present invention.

Operation

Some embodiments of the present invention provide methods of identifying preferred locations for pacing the heart. One preferred embodiment comprises navigating a pacing electrode to each of a plurality of locations in the heart; pacing the heart at each of the plurality of locations; assessing the effectiveness of the pacing at each location by ultrasonically measuring blood flow. The ultrasonically measured blood flow can be a blood flow velocity, or a blood volume. This ultrasonic measurement can be preformed with ultrasonically enabled catheters disposed in the body, or with esophageal probes, or preferably non-invasively using external ultrasound probes.

In an alternate preferred embodiment, rather than ultrasonically measuring blood flow, the blood flow is measured via medical imaging, and in particular via Magnetic Resonance (MR) imaging. From MR imaging it is also possible to measure flow volumes and cardiac strain.

The pacing electrode is navigated with the aid of a remote navigation system. The remote navigation system can be a magnetic navigation system that orients the pacing electrode through the application of a magnetic field, or it could be a mechanical navigation system that orients the pacing electrode by a mechanically actuated guiding sheath. Alternatively, the remote navigation system can be used to navigate a supporting device such as a guide wire, and the pacing lead can be tracked over the wire to a desired location. The remote navigation system may be any system for remotely orienting the distal end of a medical device disposed in an operating region in a subject, including electrostrictive, magnetostictive, pneumatic, hydraulic systems. The remote navigation system preferably also includes an advancer for advancing the pacing electrode in the direction of orientation of the distal end, although the device could be manually advanced, if desired.

The remote navigation system can be manually controlled, responding to user inputs of direction. The remote navigation system can also be semi-automatically or automatically controlled, responding to user inputs of points or preplanned patterns of points. The navigation system can also automatically determine points based at least in part upon the current location and a sensed physiologic property associated with the location. For example, the remote navigation system could receive local physiologic data (for example electrical activity) from the pacing lead, and select a new location based on the current location and its sensed physiologic properties, and/or or based upon prior locations and their sensed physiologic properties. As another example, the remote navigation system could receive information from the assessment of the effectiveness of the pacing, and select a new location based upon the current location and the assessed pacing effectiveness, and/or based upon prior locations and their assessed pacing effectiveness. Pressure-Volume data as described above constitute one form of sensed physiologic data. The use of sensed physiologic data in the control of remote navigation devices is disclosed in, U.S. Provisional Patent Application Ser. No. 60/642,853, filed Jan. 11, 2005, entitled Use of Sensed Local Physiologic Data in Positioning A Remotely Navigable Medical Device.

The system can identify the single best pacing site based on a single criterion, or based upon multiple criteria. For example, in addition to some measure of blood flow, blood pressure data, ECG data, and phrenic nerve stimulation data can be used to evaluate pacing effectiveness. When the identification is based upon multiple criteria, the various criteria can be given predetermined weights, or the weights can be adjusted by the user.

Sometimes the single best pacing site may not be desirable from some other standpoint, for example the difficulty of safely and securing affixing a pacing lead at the location. Thus the system may identify a plurality of “good” sites, e.g. a predetermined number of sites, or all sites exceeding a predetermined threshold. The user can select a site from the displayed sites. When an automated remote navigation site is used, the system can automatically return the pacing electrode to the selected site.

In accordance with another preferred embodiment of this invention, a pacing electrode is navigated to each of a plurality of locations in the heart with a remote navigation system; the heart is paced at each of the plurality of locations; and the effectiveness of the pacing at each location is assessed by ultrasonically measuring blood flow. At least some of the points are displayed in a manner that shows a measure of the assessed effectiveness, so that the user can select a point based upon its assessed effectiveness and location, and the remote navigation system automatically navigate a pacing lead to the selected location.

As with the other preferred embodiments, the blood flow can be measured ultrasonically by measuring blood flow velocity, or the blood flow volume. This ultrasonic measurement can be made with ultrasonic devices disposed inside the subject's vasculature or heart chamber, or by using a trans-esophageal ultrasound catheter, or preferably non-invasively from outside the body, using an external ultrasound transducer.

As with other preferred embodiments, the remote navigation system can be a magnetic navigation system that orients the pacing electrode through the application of a magnetic field, or it could be a mechanical navigation system that orients the pacing electrode by a mechanically actuated guiding sheath. The remote navigation system may be any system for remotely orienting the distal end of a medical device disposed in an operating region in a subject, including electrostrictive, magnetostictive, pneumatic, hydraulic systems. The remote navigation system preferably also includes an advancer for advancing the pacing electrode in the direction of orientation of the distal end, although the device could be manually advanced, if desired.

The remote navigation system can be manually controlled, responding to user inputs of direction. The remote navigation system can also be semi-automatically or automatically controlled, responding to user inputs of points or preplanned patterns of points. The navigation system can also automatically determine points based at least in part upon the current location and a sensed physiologic property associated with the location. For example, the remote navigation system could receive local physiologic data (for example electrical activity) from the pacing lead, and select a new location based on the current location and its sensed physiologic properties, and/or or based upon prior locations and their sensed physiologic properties. As another example, the remote navigation system could receive information from the assessment of the effectiveness of the pacing, and select a new location based upon the current location and the assessed pacing effectiveness, and/or based upon prior locations and their assessed pacing effectiveness.

In accordance with another preferred embodiment of this invention, a pacing electrode is navigated to a location in the heart; the heart is paced at the location; the effectiveness of the pacing at the location is assessed by ultrasonically measuring blood flow and cardiac output, and repeating these steps until a measure of the assessed effectiveness of the pacing exceeds a predetermined value.

As with the other preferred embodiments, the blood flow can be measured ultrasonically by measuring blood flow velocity, or the blood flow volume. This ultrasonic measurement can be made with ultrasonic devices disposed inside the subject's vasculature, using a trans-esophageal ultrasound catheter, or preferably non-invasively from outside the body, using an external ultrasound transducer.

As with other preferred embodiments, the remote navigation system can be a magnetic navigation system that orients the pacing electrode through the application of a magnetic field, or it could be a mechanical navigation system that orients the pacing electrode by a mechanically actuated guiding sheath. The remote navigation system may be any system for remotely orienting the distal end of a medical device disposed in an operating region in a subject, including electrostrictive, magnetostictive, pneumatic, hydraulic systems. The remote navigation system preferably also includes an advancer for advancing the pacing electrode in the direction of orientation of the distal end, although the device could be manually advanced, if desired.

In one embodiment the system includes a processor that combines pressure volume loop recording data, ECG recording data, and phrenic nerve stimulation to determine at least one optimal pacing lead placement site, and in particular lead placement sites in the left side of the heart of patients undergoing cardiac resynchronization therapy. Additional factors can be considered, including any indicator of a healthy or near-healthy cardiac cycle including Pressure-Volume loop, a narrow QRS complex in an ECG recording (in the preferred embodiment one that is less than about 120 milliseconds in width), and the absence of phrenic nerve stimulation (in the preferred embodiment with little or no respiratory disturbances from pacing) to select an acceptable lead implantation location from a set of possible locations.

What is claimed is:

1. A system for identifying locations for pacing the heart, the system comprising a pacing electrode, a remote navigation system for positioning the pacing electrode at each of a plurality of locations; a system for determining Pressure-Volume loop data resulting from pacing at each location; an ECG system, a phrenic nerve stimulation detection system, and a means of identifying at least one preferred location based upon at least the Pressure-Volume loop, ECG, and phrenic nerve stimulation data at each location.

2. The system according to claim 1 wherein the remote navigation system is a magnetic navigation system.

3. The system according to claim 1 wherein the remote navigation system is a mechanical navigation system.

4. The system according to claim 1 wherein the system for determining pressure volume loop data comprises ultrasonic imaging apparatus.

5. The system according to claim 1 wherein the system for determining pressure volume loop data incorporates a pressure transducer placed external to the patient.

6. The system according to claim 1 wherein the system for determining pressure volume loop data incorporates a pressure transducer placed internally in the patient.

7. The system according to claim 6 wherein the ultrasound imaging apparatus includes an internal ultrasound transducer.

8. The system according to claim 6 wherein the ultrasound imaging apparatus includes a trans-esophageal ultrasound transducer.

9. The system according to claim 6 wherein the ultrasound imaging apparatus includes an external ultrasound transducer.

10. The system according to claim 6 wherein the system for determining pressure volume loop data comprises a Magnetic Resonance Imaging apparatus.

11. The system according to claim 1 wherein the remote navigation system is controlled by a user to navigate the pacing electrode to each of the plurality of locations.

12. The system according to claim 1 wherein the remote navigation system has a control programmed to navigate a predetermined plurality of locations.

13. The system according to claim 1 wherein the remote navigation system has a control programmed to select at least some of the plurality of locations based upon data collected at some of the locations.

14. The system according to claim 1 wherein the remote navigation system has a control programmed to select at least some of the plurality of locations based upon the data collected upon pacing at some of the locations.

15. A method of identifying locations for pacing the heart, the method comprising:
navigating a pacing electrode to each of a plurality of locations in the heart; pacing the heart at each of the plurality of locations; assessing the effectiveness of the pacing at each location by measuring cardiac blood flow and cardiac wall strain.

16. The method according to claim 15 wherein the blood flow is measured by measuring blood flow velocity.

17. The method according to claim 15 wherein the blood flow is measured by measuring blood flow volume.

18. The method according to claim 15 wherein the blood flow is measured ultrasonically.

19. The method according to claim 15 wherein the blood flow is measured non-invasively from outside the body.

20. The method according to claim 15 wherein the pacing electrode is navigated with the aid of a remote navigation system.

21. The method according to claim 20 wherein the remote navigation system is a magnetic navigation system that orients the pacing electrode through the application of a magnetic field.

22. The method according to claim 20 wherein the remote navigation system is a magnetic navigation system that applies a magnetic field to orient a guide wire over which the pacing lead is delivered.

22. The method according to claim 20 wherein the remote navigation system is a mechanical navigation system that orients the pacing electrode by orienting a mechanically actuated guiding sheath.

23. The method according to claim 20 wherein the remote navigation system automatically navigates the pacing electrode to a plurality of locations in a preplanned pattern.

24. The method according to claim 20 wherein the remote navigation system navigates the pacing electrode to locations selected based at least in part upon the current location and a sensed physiologic property associated with the location.

25. The method according to claim 20 wherein the remote navigation system navigates the pacing electrode to locations based on the sensed physiological properties associated with previously visited locations.

26. The method according to claim 15 further comprising identifying a plurality of locations based upon the assessed effectiveness of the pacing from which the user can select a desired one.

27. The method according to claim 26 further comprising displaying the plurality of identified locations on a representation of the heart surface from which the user can select a desired one.

28. A method of identifying locations for pacing the heart, the method comprising:
navigating a pacing electrode to each of a plurality of locations in the heart; pacing the heart at each of the plurality of locations; assessing the effectiveness of the pacing at each location by a combination of measurement of cardiac blood flow data, blood pressure data, and phrenic nerve stimulation data.

29. The method according to claim 28 wherein the measurement of blood flow is done with ultrasound.

30. A method of identifying locations for pacing the heart, the method comprising:
navigating a pacing electrode to each of a plurality of locations in the heart with a remote navigation system; pacing the heart at each of the plurality of locations; assessing the effectiveness of the pacing at each location by ultrasonically measuring blood flow; displaying at least some of the points on a display in a manner that shows a measure of the assessed effectiveness, so that the user can select a point based upon its assessed effectiveness and location, and automatically navigating a pacing lead to the selected location using a remote navigation system.

31. The method according to claim 30 wherein the blood flow velocity is measured ultrasonically.

32. The method according to claim 30 wherein the blood flow volume is measured ultrasonically.

33. The method according to claim 30 wherein the blood flow is measured non-invasively from outside the body.

34. The method according to claim 30 wherein the remote navigation system is a magnetic navigation system that orients the pacing electrode through the application of a magnetic field.

35. The method according to claim 30 wherein the remote navigation system is a magnetic navigation system that applies a magnetic field to orient a guide wire over which the pacing lead is delivered.

36. The method according to claim 30 wherein the remote navigation system is a mechanical navigation system that orients the pacing electrode by a mechanically actuated guiding sheath.

37. The method according to claim wherein the remote navigation system automatically navigates the pacing electrode to a plurality of locations in a preplanned pattern.

38. The method according to claim 30 wherein the remote navigation system navigates the pacing electrode to locations selected based at least in part upon the current location and a sensed physiologic property associated with the location.

39. The method according to claim 30 wherein the remote navigation system navigates the pacing electrode to locations based on the sensed physiological properties associated with previously visited locations.

40. A method of identifying locations for pacing the heart, the method comprising: (a) navigating a pacing electrode to a location in the heart; (b) pacing the heart at the location; (c) assessing the effectiveness of the pacing at the location by ultrasonically measuring blood flow, and (d) repeating steps (a) through (c) until a measure of the assessed effectiveness of the pacing exceeds a predetermined value.

41. The method according to claim 40 wherein the measure of assessed effectiveness is based upon ultrasonically measured flow velocity.

42. The method according to claim 40 wherein the measure of assessed effectiveness is based upon ultrasonically measured flow volume.

43. The method according to claim 40 wherein the blood flow velocity is measured ultrasonically.

44. The method according to claim 40 wherein the blood flow volume is measured ultrasonically.

45. The method according to claim 40 wherein the blood flow is measured non-invasively from outside the body.

46. The method according to claim 40 wherein the pacing electrode is navigated by a remote navigation system.

47. The method according to claim 46 wherein the remote navigation system is a magnetic navigation system that orients the pacing electrode through the application of a magnetic field.

48. The method according to claim 46 wherein the remote navigation system is a magnetic navigation system that applies a magnetic field to orient a guide wire over which the pacing lead is delivered.

49. The method according to claim 46 wherein the remote navigation system is a mechanical navigation system that orients the pacing electrode by a mechanically actuated guiding sheath.

50. The method according to claim 46 wherein the remote navigation system automatically navigates the pacing electrode to a plurality of locations in a preplanned pattern.

51. The method according to claim 46 wherein the remote navigation system navigates the pacing electrode to locations selected based at least in part upon the current location and a sensed physiologic property associated with the location.

52. The method according to claim 46 wherein the remote navigation system navigates the pacing electrode to locations based on the sensed physiological properties associated with previously visited locations.

53. A method of identifying locations for pacing the heart, the method comprising:
navigating a pacing electrode to each of a plurality of locations in the heart; pacing the heart at each of the plurality of locations; assessing the effectiveness of the pacing at each location by a combination of measurement of phrenic nerve stimulation data and ECG data.

54. The method according to claim 53 where the pacing electrode is navigated using a remote navigation system.

55. The method according to claim 54 wherein the remote navigation system is a magnetic navigation system that orients the pacing electrode through the application of a magnetic field.

56. The method according to claim 54 wherein the remote navigation system is a magnetic navigation system that applies a magnetic field to orient a guide wire over which the pacing lead is delivered.

57. The method according to claim 54 wherein the remote navigation system is a mechanical navigation system that orients the pacing electrode by a mechanically actuated guiding sheath.

58. The method according to claim 54 wherein the remote navigation system automatically navigates the pacing electrode to a plurality of locations in a preplanned pattern.

59. The method according to claim 54 wherein the remote navigation system navigates the pacing electrode to locations selected based at least in part upon the current location and a sensed physiologic property associated with the location.

60. The method according to claim 54 wherein the remote navigation system navigates the pacing electrode to locations based on the sensed physiological properties associated with previously visited locations.
